SEQUENCE LISTING (7 sequences)

SEQ ID NO: 1 - DNA, 41 bp, H. sapiens
ggccccgccctctcctcgcggcgcgagtttcaggcagcgct

SEQ ID NO: 2 - DNA, 13 bp, H.sapiens
ggcgcgagtttca

SEQ ID NO: 3 - DNA, 10 bp, H.sapiens
cgcgagtttc

SEQ ID NO: 4 - DNA, 21 bp, H.sapiens
ggcgcgagtttcaggcagcgc

SEQ ID NO: 5 - DNA, 10 bp, H. Sapiens
cgcgagtttc

SEQ ID NO: 6 - DNA, 144 bp, H. Sapiens
agcaaccatagtcccgcccctaactccgcccatcccgcccctaactccgcccagttccgcccattctccgccccatcgctgaggcgcgagtttcaggcagcgcgaggccgaggccgcctcggcctctgagctattccagaagta

SEQ ID NO: 7 - DNA, 144 bp, H. sapiens
cccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccac

(19) United States
(12) Patent Application Publication
(10) Pub. No.: US 2003/0113760 A1
(43) Pub. Date: Jun. 19, 2003

(54) Methods and compositions for use in selectively producing a protein in telomerase expressing cells

(76) Inventors: William H. Andrews, Reno, NV (US); Daniel Fylstra, Incline Village, NV (US); Christopher A. Foster, Carmichael, CA (US); Stephanie Fraser, Sparks, NV (US); Hamid Mohammadpour, Reno, NV (US); Laura Briggs, Reno, NV (US)

Correspondence Address: BOZICEVIC, FIELD & FRANCIS LLP, 200 MIDDLEFIELD RD, SUITE 200, MENLO PARK, CA, 94025, US

(21) Appl. No.: 10/219,450

(22) Filed: Aug. 13, 2002

Related U.S. Application Data
(60) Provisional application No. 60/313,238, filed on Aug. 17, 2001.

(51) Int. Cl. C12Q 1/68; C12P 21/02; C12N 5/06

(52) U.S. Cl. 435/6; 435/69.1; 435/320.1; 435/325

(57) ABSTRACT

Methods and compositions for use in selectively expressing a protein in a telomerase expressing cell are provided. In the subject methods, an expression cassette comprising a Site C repressor site and a coding sequence for the protein is introduced into the target telomerase expressing cell, e.g., by administering the expression cassette to a host that includes the target cell. The protein may be a therapeutic or diagnostic protein. The subject methods find use in a variety of different applications, and are particularly suited for use in diagnostic and therapeutic applications, e.g., of cellular proliferative disease conditions.

### CROSS-REFERENCE TO RELATED APPLICATIONS

Pursuant to 35 U.S.C. § 119 (e), this application claims priority to the filing date of the U.S. Provisional Patent Application Ser. No.: (a) 60/313,238 filed Aug. 17, 2001; the disclosure of which is herein incorporated by reference.

### FIELD OF THE INVENTION

The field of this invention is the treatment of cellular proliferative disease conditions, e.g., cancer.

### BACKGROUND OF THE INVENTION

There is much interest in the development of effective treatments for cancer. Designing effective treatments for patients with cancer has represented a major challenge. The current regimen of surgical resection, external beam radiation therapy, and/or systemic chemotherapy has been partially successful in some kinds of malignancies, but has not produced satisfactory results in others.

In contrast to conventional systemic cytokine- or chemotherapy, gene therapy is based on the introduction of deoxyribonucleic acid (DNA) into the tumor cells, surrounding parenchyma or cells involved in the antitumoral immune response. The integrated DNA encodes for cytokines or enzymes that will ultimately result in tumor cell death. Gene transfer is rapidly becoming a useful adjunct in the development of new therapies for human malignancy. Theoretically, the most direct mechanism for tumor cell killing using gene transfer is the selective expression of cytotoxic gene products within tumor cells. Unlike gene therapies that correct a genetic aberration responsible for the cancer, the suicide gene strategy involves a gene that is unrelated to human cancer, such as the Herpes simplex thymidine kinase gene. If properly inserted into human cancer cells, the gene changes the DNA of the cells so that they become susceptible to the antiviral drug ganciclovir. In addition to the Herpes simplex virus thymidine kinase (HSV-tk) gene, other genes have also been employed in suicidal gene therapy protocols, including, but not limited to: caspase 6 and 8, FADD, Bax, etc.

In suicide gene therapy, it is desirous to limit expression of the suicide gene to disease cells. As such, there is continued interest in this area of gene therapy for the identification of gene therapy vectors and approaches employing the same that provide for exclusive expression of therapeutic suicide genes in cancer cells. The present invention satisfies this need.

Relevant Literature

Patent publications of interest include: WO 02/16657 and WO 02/16658 and the references cited therein. Also of interest are: Gu et al., Gene Ther. (2002) 9:30-37; Gu et al., Cancer Res. (2000) 60:5359-5364; Koga et al., Hum. Gene Ther. (2000) 11:1397-406; Koga et al., Anticancer Res. (2001) 21:1937-1943; Komata et al., Cancer Res. (2001) 61:5796-802; Komata et al., Int. J. Oncol. (2001) 19:1015-1020; and Majumdar et al., Gene Ther. (2001) 8:568-578.

### SUMMARY OF THE INVENTION

Methods and compositions for use in selectively expressing a protein in a telomerase expressing cell are provided. In the subject methods, an expression cassette comprising a Site C repressor site and a coding sequence for the protein is introduced into the target telomerase expressing cell, e.g., by administering the expression cassette to a host that includes the target cell. The protein may be a therapeutic or diagnostic protein. The subject methods find use in a variety of different applications, and are particularly suited for use in diagnostic and therapeutic applications, e.g., of cellular proliferative disease conditions.

### DESCRIPTION OF THE SPECIFIC EMBODIMENTS

Methods and compositions for use in selectively expressing a protein in a telomerase expressing cell are provided. In the subject methods, an expression cassette comprising a Site C repressor site and a coding sequence for the protein is introduced into the target telomerase expressing cell, e.g., by administering the expression cassette to a host that includes the target cell. The protein may be a therapeutic or diagnostic protein. The subject methods find use in a variety of different applications, and are particularly suited for use in diagnostic and therapeutic applications, e.g., of cellular proliferative disease conditions.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the cell lines, vectors, and methodologies which are described in the publications which might be used in connection with the presently described invention.

In further describing the subject invention, the subject methods and compositions are described first in greater detail, followed by a discussion of various representative applications in which the subject methods and compositions find use as well as a review of kits that find use in practicing the subject methods.

Methods and Compositions

As summarized above, the subject invention provides methods and compositions for selectively expressing a protein of interest in a telomerase producing cell. As such, the subject invention provides methods and compositions for selectively expressing a protein of interest in a cell that expresses telomerase, where the telomerase expressing cell may or may not be present in a collection of cells, some of which may not express or produce telomerase. In practicing the subject methods, an expression cassette that includes a promoter and coding sequence for the protein of interest operatively linked to at least one Site C repressor site is introduced into the target telomerase expressing cell, resulting in production of the protein of interest in the target cell.

The target telomerase expressing cell may be a number of different types of cells, including: a cell in vitro, e.g., isolated or together with other cells, e.g., in a cell culture, as part of a tissue sample, organ, etc., separated from its host, and the like; a cell in vivo, e.g., a cell present in a host, etc. As such, the telomerase expressing cell may be in culture with other telomerase expressing cells, non-telomerase expressing cells or a combination thereof. Alternatively, the target cell may be present as an individual cell or collection of cells, where the collection of cells may be a whole, multicellular animal or portion thereof, e.g., tissue, organ, etc. As such, the target cell(s) may be in a host animal or portion thereof, or may be a therapeutic cell (or cells) which is to be introduced into a multicellular organism, e.g., a cell employed in gene therapy. The target cell within a host may be within a tissue or population of cells comprising other telomerase expressing cells, non-telomerase expressing cells or a mixture thereof.

As the target cell is a telomerase expressing/producing cell, the target cell is one that lacks a functional Site C repressor system (e.g., a single protein or plurality of proteins acting in concert) that interacts with a Site C repressor site (e.g., by binding to the site) in the telomerase minimal promoter to inhibit telomerase expression. In other words, the target cell is a cell in which a functional Site C repressor system is not present or is so minimally active as to not substantially inhibit telomerase expression.

In practicing the subject methods, an expression cassette that includes a coding sequence for the protein of interest operably linked to a promoter and a Site C repressor sequence/site/domain is introduced into the target cell. By expression cassette (i.e., expression system) is meant a nucleic acid molecule that includes a promoter and a Site C site/domain operably linked to a sequence encoding a peptide or protein of interest, i.e., a coding sequence, where by operably linked is meant that expression of the coding sequence is modulated by the Site C sequence and interactions at the Site C sequence, e.g., binding at the Site C sequence inhibits expression of the coding sequence.

The Site C sequence of the expression cassettes employed in the subject methods is a nucleic acid sequence identical or substantially similar to a sequence/domain/region of the minimal tert promoter that binds a Site C tert expression repression system, e.g., a transcription factor or collection of factors that inhibits tert expression by binding to a Site C sequence/domain of the minimal tert promoter. Any nucleic acid sequence that is capable of binding to the Site C tert expression repression system and thereby inhibiting expression of the coding sequence to which it is operably linked may be employed.

The Site C domain present in the subject expression vectors typically ranges in length from about 1 base, usually at least about 5 bases and more usually at least about 15 bases, to a length of about 25 bases or longer. In many embodiments, the length of the subject Site C site/domain ranges in length from about 1 to about 50 bases, usually from about 5 to about 45 bases. A feature of the subject invention is that the Site C domain is not present in the Tert minimal promoter, such that it is separate from its naturally occurring environment. As such, the expression cassettes employed in the subject methods do not include a TERT minimal promoter sequence, e.g., the human 378 bp TERT core promoter as described in Takakura et al., Cancer Res. (1999)59:551-557. Since the Site C sequence is not present with its entire core promoter, it is also viewed as a non-naturally occurring, synthetic, isolated Site C sequence.

In many embodiments, the Site C site has a sequence found in a limited region of the human tert minimal promoter, where this limited region typically ranges from about −40 to about −90, usually from about −45 to about −85 and more usually from about −45 to about −80 relative to the “A” of the telomerase ATG codon.

Of particular interest in certain embodiments is a nucleic acid having a sequence found in SEQ ID NO: 01 (e.g., a sequence range of at least about 2, usually at least about 5 and often at least about 10, 20, 25, 30 or more bases up to about 45 to 50 bases, where, in certain embodiments, the Site C domain will have a sequence that is identical to a sequence of SEQ ID NO: 01. SEQ ID NO: 01 has the following sequence:

GGCCCCGCCCTCTCCTCGCGGCGCGAGTTTCAGGCAGCGCT (SEQ ID NO: 01)

In certain embodiments, the Site C site includes the sequence of −69 to −57 of the human tert minimal promoter. In other words, the sequence of the Site C site is:

GGCGCGAGTTTCA (SEQ ID NO: 02).

In certain embodiments, the Site C site includes the sequence of −67 to −58 of the human tert minimal promoter. In other words, the sequence of the Site C site is:

CGCGAGTTTC (SEQ ID NO: 03).

In certain embodiments, the Site C site includes the sequence of −69 to −49 of the human tert minimal promoter. In other words, the sequence of the Site C site is:

GGCGCGAGTTTCAGGCAGCGC (SEQ ID NO: 04).

The Site C site or domain employed in the subject expression cassettes may be identical to or substantially similar to the above specified Site C sequences. A given sequence is considered to be substantially similar to one of the above specific sequences if it shares high sequence similarity with the above described specific sequence, e.g. at least 75% sequence identity, usually at least 90%, more usually at least 95% sequence identify with the above specific sequence. Sequence similarity is calculated based on a reference sequence, which may be a subset of a larger sequence. A reference sequence will usually be at least about 6 nt long, more usually at least about 8 nt long, and may extend to the complete sequence that is being compared. Algorithms for sequence analysis are known in the art, such as BLAST, described in Altschul et al. 108(1990), J. Mol. Biol. 215:403-10. Unless otherwise noted, the above algorithm set at default settings is employed to determine sequence identity.

Of particular interest are Site C nucleic acids of substantially the same length as the specific nucleic acid sequences identified above, where by substantially the same length is meant that any difference in length does not exceed about 20 number %, usually does not exceed about 10 number % and more usually does not exceed about 5 number %. In these embodiments, the Site C domains have sequence identity to one of the above described sequences of at least about 90%, usually at least about 95% and more usually at least about 99% over the entire length of the nucleic acid.

The Site C site/domain present in the subject expression cassettes may have one or more modifications with respect to the above described specific sequences, where such modifications include sequence mutations, deletions, and insertions, so long as the modified Site C domain is functional for its intended purpose, e.g., to bind to telomerase repression systems in cells that do not express telomerase by action of such repression systems. As such, these modified Site C sequences retain the functional property of the Site C binding site sequence, namely, they will still permit the repression of the expression of the protein of interest in cells containing a functional Site C repressor system (e.g. normal cells), while allowing expression of the protein of interest in cells where a functional Site C repressor system is absent or minimally operative such that telomerase is expressed (e.g. cells associated with proliferative diseases).

The number of Site C sites/domains may vary, where a single Site C site may be present on the expression cassette or a plurality of Site C sites may be present, where when a plurality are present, the number typically ranges from about 2 to about 10, more usually from about 2 to about 5, where in certain embodiments the Site C domains/sequences may or may not be separated by intervening domains or spacers of from about 2 to about 10 nt in length, usually from about 2 to about 5 nt in length.

The Site C domain of the human tert minimal promoter is further described in U.S. patent application Ser. Nos. 60/227,865; 60/230,174; 60/238,345 and 60/275,681; the disclosures of which are herein incorporated by reference.

In addition to the Site C site/domain described above, the expression vectors employed in the subject methods also generally include a coding sequence for a protein of interest, where the protein of interest may be a therapeutic protein or a marker protein, depending on the particular application for which the subject method is being performed, as described in greater detail below.

In certain embodiments, the coding sequence of the subject expression vector encodes a therapeutic protein that, when expressed in a target telomerase producing/expressing cells, inhibits cell growth and/or induces cell death. Suitable coding sequence of interest include, but are not limited to, coding sequences for enzymes, tumor suppressor proteins, toxins, cytokines, apoptosis proteins, and the like. Representative enzymes of interest as therapeutic proteins include thymidine kinase (TK), xanthineguanine phosphoribosyltransferase (GPT), cytosine deaminase (CD), hypoxanthine phosphoribosyl transferase (HPRT), E. coli. purine nucleoside phosphorylase (PNP), and the like. Representative tumor suppressor proteins include neu, EGF, ras (including H, K, and N ras), p53 retinoblastoma tumor suppressor gene (Rb), Wilm's Tumor Gene Product, Phosphotyrosine Phosphatase (PTPase), and nm 23. Representative toxins include Pseudomonas exotoxin A and S; diphtheria toxin (DT); E. coli LT toxins, Shiga toxin, Shiga-like toxins (SLT1, -2), ricin, abrin, supporin, and gelonin. Representative cytokines include interferons, interleukins, tumor necrosis factor (TNF), and the like. Representative apoptosis proteins of interest include Bax, Caspase-8, FADD (Fas-associated death domain) and the like. The proteins and genes of interest described above are only exemplary of the types of proteins useful in inhibiting and killing telomerase expressing cells. By no means are the above examples to be limiting to the scope of the subject invention.

Instead of therapeutic proteins, such as the suicide genes described above, the coding sequence may be a coding sequence for a marker/diagnostic protein. Marker proteins of interest include proteins that code for a product that is either directly or indirectly detectable. Directly detectable proteins of interest for use as marker proteins include fluorescent proteins. A large number of different fluorescent proteins are known to those of skill in the art and include, but are not limited to: green fluorescent proteins from Aequoria victoria, fluorescent proteins from non-bioluminescent anthozoa species, as well as homologs, mutants and mimetics therefor. U.S. Pat. Nos. disclosing green fluorescent proteins and mutants/homologs thereof include: 5,491,084; 5,625,048; 5,741,668; 5,795,737; 5,804,387; 5,874,304; 5,968,750; 6,020,192; 6,077,707; 6,027,881; 6,124,128; 6,146,826; and the like. The disclosures of these patents are herein incorporated by reference. Fluorescent proteins from non-bioluminescent species of interest include, but are not limited to, those described in the following PCT published applications: WO 00/34318; WO 00/34320; WO 00/34321; WO 00/34322; WO 00/34319; WO 00/34323; WO 00/34526; WO 00/34324; WO 00/34325; WO 00/34326; WO 01/27150; the disclosures of the priority documents of which are herein incorporated by reference. Indirectly detectable marker proteins of interest include proteins that interact with one or more members of a signal producing system to produce a detectable product. Representative indirectly detectable proteins of interest include, but are not limited to: enzymes that convert a substrate to a detectable product, e.g., luciferase, and the like.

In addition to the above described Site C binding site and coding sequences, the subject expression cassette typically further includes a promoter sequence that drives expression of the coding sequence in the absence of a Site C repressor system. A number of different promoter sequences suitable for use in the subject expression vectors are known, where representative promoter sequences of interest include CMV promoter, SV40 promoter, and the like. In certain embodiments, the promoter is not a Tert promoter, such as the human Tert minimal promoter or functional portion thereof. The promoter system, Site C domain and coding sequence are all operably linked on the expression vector such that in the absence of the Site C repressor system in the target host cell, the promoter drives expression of the coding sequence but in the presence of the repressor system, the coding sequence is not expressed.,

In certain embodiments, the coding sequence for the protein of interest is flanked by endonuclease recognized sites, i.e., a restriction sites, which may or may not be part of a multiple cloning site. A variety of restriction sites are known in the art and may be included in the expression cassette, where such sites include those recognized by the following restriction enzymes: HindIII, PstI, SaII, AccI, HincII, XbaI, BamHI, SmaI, XmaI, KpnI, SacI, EcoRI, and the like. In many embodiments, the expression cassette includes a polylinker, i.e., a closely arranged series or array of sites recognized by a plurality of different restriction enzymes, such as those listed above. As such, in many embodiments, the expression cassettes include a multiple cloning site made up of a plurality of restriction sites. The number of restriction sites in the multiple cloning site may vary, ranging anywhere from 2 to 15 or more, usually 2 to 10.

Construction of expression cassettes suitable for the subject invention may be done using standard ligation and restriction techniques, which are well understood in the art (see Maniatis et al., (1982) in Molecular Cloning: A Laboratory Manual, Cold Spring Harbor Laboratory, New York). Isolated plasmids, DNA sequences, or synthesized oligonucleotides are cleaved, tailored, and religated in the form desired expression cassettes which include a gene coding for the protein of interest, and control sequences such as a promoter and the Site C sequence.

In practicing the subject invention, an effective amount of the above described expression vectors are introduced into the target cell in a manner such that, in the absence of a Site C repressor system, the coding sequence of the expression cassette is expressed in the target cell. Any convenient manner of introducing the expression cassette into the target cell may be employed, where a number of different protocols are known to those of skill in the art. Determination of an effective amount necessarily depends on the particular application being performed, and can readily be determined empirically. An effective amount is any amount that is sufficient to achieve the intended purpose, e.g., therapeutic, diagnostic, etc.; as described below.

In many embodiments, it is desirable to employ a vector to deliver the expression cassette to the interior of the target cell. Vectors of interest include, but are not limited to: plasmids; viral vectors, e.g., lentivirus, adenovirus, adeno-associated virus, vaccinia virus, herpes virus, rabies virus, Moloney murine leukemia virus, papovavirus, JC, SV40, polyoma, Epstein-Barr Virus, papilloma virus; and the like, where the vectors are able to transiently or stably be maintained in the cells, usually for a period of at least about one day, more usually for a period of at least about several days to several weeks. The choice of appropriate vector is well within the skill of the art and many vectors useful in the subject invention are available commercially.

The expression cassette or vector including the same may be introduced into the target cell using any convenient protocol, where the protocol may provide for in vitro or in vivo introduction of the expression cassette. A number of different in vitro protocols exist for introducing nucleic acids into cells, and may be employed in the subject methods. Suitable protocols include: calcium phosphate mediated transfection; DEAE-dextran mediated transfection; polybrene mediated transfection; protoplast fusion, in which protoplasts harboring amplified amounts of vector are fused with the target cell; electroporation, in which a brief high voltage electric pulse is applied to the target cell to render the cell membrane of the target cell permeable to the vector; liposome mediated delivery, in which liposomes harboring the vector are fused with the target cell; microinjection, in which the vector is injected directly into the cell, as described in Capechhi et al, Cell (1980) 22:479; and the like. The above in vitro protocols are well known in the art and are reviewed in greater detail in Sambrook, Fritsch & Maniatis, Molecular Cloning: A Laboratory Manual (Cold Spring Harbor Laboratory Press) (1989) pp 16.30-16.55.

Where introduction is to be carried out in vivo, contact is generally achieved by administering a suitable preparation of the expression cassette to the organism in which the target or host cell is located, e.g. to the multicellular organism. Any convenient mode of administration may be employed. In many embodiments, intravascular methods of administration are employed, e.g. intra-arterial, intravenous, etc., where intravenous administration is preferred in many embodiments. In vivo protocols that find use in delivery of the subject vectors also include delivery via lipid based, e.g. liposome vehicles, where the lipid based vehicle may be targeted to a specific. cell type for cell or tissue specific delivery of the vector. Patents disclosing such methods include: U.S. Pat. Nos. 5,877,302; 5,840,710; 5,830,430; and 5,827,703, the disclosures of which are herein incorporated by reference. Other in vivo delivery systems may also be employed, including: the use of polylysine based peptides as carriers, which may or may not be modified with targeting moieties, microinjection, electroporation, and the like. (Brooks, A. I., et al. 1998, J. neurosci. Methods V. 80 p: 137-47; Muramatsu, T., Nakamura, A., and H. M. Park 1998, Int. J. Mol. Med. V.1 p: 55-62). Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells.

The amount of vector nucleic acid that is introduced into the cell is sufficient to provide for the desired expression of the encoded protein. As such, the amount of vector nucleic acid introduced should provide for a sufficient amount encoded protein product. The amount of vector nucleic acid that is introduced into the target cell varies depending on the efficiency of the particular introduction or transfection protocol that is employed.

The above described methods result in expression of the coding sequence of the protein in a cell that does not have a functional Site C repressor system, as described above. However, when the above methods are employed to introduce the subject expression cassettes into cells that do include a functional Site C repressor system, the protein coding sequence of the expression cassette is not expressed. As such, the subject methods are methods for selectively expression a protein of interest in cells that lack a functional Site C repressor system. The methods may be in vitro or in vivo, as described above, and may be used to selectively express the protein of interest in a cell that is a member of a homogeneous or heterogeneous population of cells with respect to telomerase expression.

It should be noted that while the above discussion is provided for clarity in terms of targeting a single cell, in certain embodiments a plurality of cells are targeted in a given method, depending on the particular application, where by plurality is meant at least 2, e.g., 5, 10, 50, 100, 1000, 10000, 100000, etc.

Utility

The subject methods find use in any application in which ectopic expression of an introduced coding sequence in a telomerase expressing/producing cell is desired. As such, the subject methods find use in research applications. Examples of research applications in which the subject methods find use include applications designed to characterize a particular gene. In such applications, the expression cassette is employed to insert a gene of interest into a target telomerase producing cell and the resultant effect of the inserted gene on the cell's phenotype is observed. In this manner, information about the gene's activity and the nature of the product encoded thereby on the telomerase producing cell can be deduced.

In addition to the above research applications, the subject vectors also find use in the synthesis of polypeptides, e.g. proteins of interest. In such applications, a vector that includes a gene encoding the polypeptide of interest in combination with requisite and/or desired expression regulatory sequences, e.g. promoters, etc., (i.e. an expression module) is introduced into the target telomerase producing cell that is to serve as an expression host for expression of the polypeptide. Following introduction and subsequent stable integration into the target cell genome, the targeted host cell is then maintained under conditions sufficient for expression of the integrated gene. Once the transformed host expressing the protein is prepared, the protein is then purified to produce the desired protein comprising composition. Any convenient protein purification procedures may be employed, where suitable protein purification methodologies are described in Guide to Protein Purification, (Deuthsered.) (Academic Press, 1990). For example, a lysate may be prepared from the expression host expressing the protein, and purified using HPLC, exclusion chromatography, gel electrophoresis, affinity chromatography, and the like.

The subject methods also find use in therapeutic applications in which it is desired to selectively express a therapeutic protein in a telomerase producing/expressing cell, where, in many embodiments, the target cell(s) is present in a collection of cells, at least some of which do not express telomerase. A representative therapeutic application which it is desired to selectively express a therapeutic protein in a telomerase producing/expressing cell is the treatment of cellular proliferative disease conditions, e.g., cancers and related conditions characterized by abnormal cellular proliferation concomitant with the presence of telomerase expression and activity. Such disease conditions include cancer/neoplastic diseases and other diseases characterized by the presence of unwanted cellular proliferation, e.g., hyperplasias, where such conditions are described in, for example, U.S. Pat. Nos. 5,645,986; 5,656,638; 5,703,116; 5,760,062; 5,767,278; 5,770,613; and 5,863,936; the disclosures of which are herein incorporated by reference. Representative therapeutic genes of interest for use in such applications include those listed above. Depending on the nature of the therapeutic gene, one or more additional agents that work in concert with the therapeutic gene may be contacted with the cell to achieve the desired effect. As such, the methods of the present invention can provide a highly general method of treating many—if not most—malignancies, as demonstrated by the highly varied human tumor cell lines and tumors having telomerase activity, including tumors derived from cells selected from skin, connective tissue, adipose, breast, lung, stomach, pancreas, ovary, cervix, uterus, kidney, bladder, colon, prostate, central nervous system (CNS), retina and blood, and the like. More importantly, the subject methods can be effective in providing treatments that discriminate between malignant and normal cells to a high degree, avoiding many of the deleterious side-effects present with most current chemotherapeutic regimes which rely on agents that kill dividing cells indiscriminately as well as tissue specific gene therapy utilizing known suicide genes. Cancers known to have increased telomerase expression associated with malignant growth and abnormal cellular proliferation include, but are not limited to: Head/Neck and Lung tissue (e.g., Head and neck squamous cell carcinoma, Non-small cell lung carcinoma, Small cell lung carcinoma) Gastrointestinal tract and pancreas (e.g., Gastric carcinoma, Colorectal adenoma, Colorectal carcinoma, Pancreatic carcinoma); Hepatic tissue (e.g:, Hepatocellular carcinoma), Kidney/urinary tract (e.g., Dysplastic urothelium, Bladder carcinoma, Renal carcinoma, Wilms tumor) Breast (e.g., Breast carcinoma); Neural tissue (e.g., Retinoblastoma, Oligodendroglioma, Neuroblastoma, Meningioma malignant; Skin (e.g., Normal epidermis, Squamous cell carcinoma, Basal cell carcinoma, Melanoma, etc.); Hematological tissues (e.g., Lymphoma, CML chronic myeloid leukemia, APL acute promyelocytic leukemia, ALL acute lymphoblastic leukemia, acute myeloid leukemia, etc.).

The subject methods also find use in diagnostic applications in which the identification of telomerase producing cells in a collection of cells is desired. Specifically, the subject methods find use in both in vitro and in vivo diagnostic procedures for the ready identification of telomerase producing cells and disease conditions associated with the presence thereof, e.g., cellular proliferative disease conditions, etc. In representative in vitro diagnostic procedures, a sample of interest is obtained and contacted with an expression system that includes a coding sequence for a marker protein, as described above, in a manner such that the expression system is taken up by the cells in the sample. The cells in the sample are then screened to detect the marker gene. Expression of the marker gene, e.g., as detected through fluorescence detection, indicates that the particular cell is a telomerase expressing/producing cell. In representative in vivo diagnostic procedures, the expression cassette is introduced into the target cells in vivo and those cells that express telomerase are detected by detecting the presence of the encoded marker protein. A representative specific application of interest is in the diagnosis of metastisis.

A variety of hosts are treatable according to the subject methods. Generally such hosts are “mammals” or “mammalian,” where these terms are used broadly to describe organisms which are within the class mammalia, including the orders carnivore (e.g., canine and feline),equine, porcine, bovine; avian, rodentia (e.g., mice, guinea pigs, and rats), and primates (e.g., humans, chimpanzees, and monkeys). In many-embodiments, the hosts will be humans.

Kits

Also provided are kits for use in practicing the subject methods. The subject kits at least include an expression cassette as described above, where the expression cassette may or may not include a coding sequence for a protein of interest, depending on whether the user of the kit desires the ability to customize the expression cassette to include a particular coding sequence of interest. As such, in certain embodiments, the kits include a complete expression cassette that includes a coding a sequence, e.g., for a therapeutic or diagnostic protein, and are employed by the end user without modification/customization. Alternatively, the kits may include an expression cassette that lacks a protein coding sequence, and optionally reagents for use in customization of the expression cassette depending on the particular intended application, where reagents of interest include restriction enzymes, one or more different protein coding sequences, etc. For example, a kit could contain an expression cassette having a multiple cloning site, one or more restriction enzymes and one or more coding sequences for different therapeutic proteins, and the end user could then customize the expression cassette to include a particular protein best suited for use in the application to be performed with the expression cassette. The various components of the kit may be present in separate containers or certain compatible components may be precombined into a single container, as desired.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is, printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXPERIMENTAL

### I. Deletion Experiments To Identify Site C

118 deletions of the minimal telomerase promoter (as defined by Takahura et al., Cancer Res. (1999) 59:551-7) were constructed to find regions within the telomerase promoter that contain potential repressor sites. These deletions ranged in size from 10 to 300 bases. Each deletion version of the minimal promoter was tested for its ability to express SEAP in MRC5 and HELA cells. Several of the deletions, all mapping about 50-100 bases upstream of the telomerase translation initiation codon (ATG), showed ˜10 fold increased expression. The highest expression in MRC5 was obtained with the deletion called 11K. This 30 base deletion includes bases −48 to −77 relative to the translation initiation codon ATG. However, a similar deletion, called 12 K, that includes bases −48 to −57 results in 500 fold less expression. On the other hand, when 11K and 12K were compared in HELA, they both gave equivalent amounts of expression. The repressor site in this region of the TERT minimal promoter therefore is contained, or overlaps with, the 20 bases present in 12 K and absent in 11 K (i.e. −58 to −77).

To identify more specifically the bases that make up this repressor site, additional deletions were made. Each deletion is 10 bases long with 7 to 8 base overlaps between consecutive deletions. The deletions were made in the minimal telomerase promoter in pSS120. Each deletion mutant was independently made three times and all deletions were transiently transfected into MRC5 (telomerase negative normal cells) and HELA (telomerase positive immortal cells).

A portion of the 5′ untranslated region is shown below, from −77 to 1, the start of translation (SEQ ID No: 2). The Site C repressor site extends from −69 to −58, as shown.

| | |
|---|---|
| CTCCTCGC GGCGCGAGTT TCAGGCAGCG CTGCGTCCTG CTGCGCACGT GGGAAGCCCT | SEQ ID NO:2 |
| r\overline{epressor site} (−69 to −58) | |
| GGCCCCGGCC ACCCCCGCGA | |
| \| | |
| start codon (1) | |

The repressor site is provided separately below as SEQ ID NO. 1.

SEQ ID NO: 1 GGCGCGAGTTTC

The expression levels were measured using the Secreted Alkaline Phosphatase Assay (SEAP) system commercially available from Clontech Laboratories, Inc. (Palo Alto, Calif.). The results are shown below.

| Deletion | MRC5 | HELA |
|---|---|---|
| NONE (control) | 0.1931 | 78.3076 |
| −104 to −95 | 0.19 | 78.30 |
| −102 to −93 | 4.92 | 73.97 |
| −99 to −90 | 1.19 | 86.95 |
| −97 to −88 | 1.69 | 97.94 |
| −94 to −85 | 8.06 | 89.6 |
| −92 to −83 | 7.89 | 89.86 |
| −89 to −80 | 12.00 | 93.91 |
| −87 to −78 | 7.26 | 59.74 |
| −84 to −75 | 7.77 | 85.48 |
| −82 to −73 | 4.83 | 99.4 |
| −79 to −70 | 3.79 | 73.34 |
| −77 to −68 | 17.15 | 82.26 |
| −74 to −65 | 34.44 | 78.99 |
| −72 to −63 | 33.22 | 123.8 |
| −69 to −60 | 33.15 | 133.56 |
| −67 to −58 | 56.98 | 97.74 |
| −64 to −55 | 21.82 | 127.32 |
| −62 to −53 | 4.60 | 108 |
| −59 to −50 | 19.58 | 103.1 |

The column of deletions indicates the bases that were deleted in the repressor site, which is indicated relative to the AUG start codon. The columns for MRC5 and HELA show the level of expression observed for each deletion, reported as a percentage of the SV40 early promoter, which was used to normalize the two cell lines.

The data demonstrate that the deletion from “−67 to −58” gave a reading of 56.9852, as compared to a reading of 0.193109 in the control cells with no deletion in the promoter, giving an increase of 295 fold higher expression. This same deletion gave only 97.746 in HELA cells, compared to the undeleted control value of 78.3076, resulting in a 1.25 fold higher expression. This finding indicates that a repressor function operates in MRC5 cells to repress expression of the wild type telomerase promoter. When the expression level of deletion “−67 to −58” in MRC5 is compared to the wild type promoter in HELA it is observed that the deletion resulted in almost as much expression as the levels observed in HELA that are sufficient to maintain telomere length. That is, the expression of the deletion in MRC5 was 59.9852/78.3076=77% of the wild type in HELA. This finding indicates that depressing the repressor in MRC5 allows for sufficient amounts of telomerase expression to maintain the length of the telomeres in the cells during cell division, and to stop cellular aging in these cells.

### II. Fine Mapping of the Site C Site

A “fine mapping” analysis of the Site C binding site was completed to determine the effect of each base within site C on telomerase repression and the results are tabulated below and shown graphically in FIG. 3. The “fine mapping” analysis involved single base mutations or deletions within Site C and assayed for their affects on the TERT promoter's ability to drive the expression of the SEAP reporter gene in transient transfection assays. In the graph of FIG. 3 the letters on the X-axis labeled “before” are the bases of Site C before mutagenesis. The letters labeled “after” are what the bases were changed to by in vitro mutagenesis. In this experiment only one base was changed at a time. That is, in one plasmid the C at −70 was changed to an A. That was the only change that took place in the plasmid. In another plasmid A at −63 was changed to a T. Again, that was the only change that took place in the plasmid. Each plasmid was then transiently transfected into MRC5 cells and expression of SEAP was assayed. The first data point shows the expression of SEAP under control of the wild type telomerase minimal promoter. This shows almost zero (83.10 SEAP units) expression. The next data point shows SEAP expression when the entire 10 base Site C sequence (SEQ ID NO. 03) is deleted. All the subsequent data points show the expression resulting from each of the single base changes shown in the X-axis.

This analysis resulted in the identification of the specific bases within site C that control the regulation of the telomerase promoter. Bases within the site C repressor binding site which were found to be influential in telomerase repression are shown in the site C sequence below as capital letters while those bases when mutated or deleted had little or no effect on telomerase repression are shown in small case.

Site C “fine mapping” results—CGCGagtTTc SEQ ID NO. 05

These results also show that the sequence that the Site C binding protein binds to is GGCGCGAGTTTCA (SEQ ID NO: 02).

| Plasmid | Base # | Mutation | SEAP |
|---|---|---|---|
| pSSl20 | | Wild Type | 83.10 |
| pSSl304 | | −67 to −58 deleted | 3093.70 |
| pSSl658 | −72 | C−>G | 268.37 |
| pSSl663 | −71 | G−>A | 208.63 |
| pSSl664 | −70 | C−>A | 256.93 |
| pSSl667 | −69 | G−>C | 596.70 |
| pSSl552 | −68 | G−>C | 879.20 |
| pSSl645 | −67 | C−>G | 1841.70 |
| pSSl670 | −66 | G−>C | 3021.37 |
| pSSl673 | −65 | C−>A | 3274.37 |
| pSSl677 | −64 | G−>A | 2115.03 |
| pSSl679 | −63 | A−>T | 968.70 |
| pSSl682 | −62 | G−>C | 542.80 |
| pSSl686 | −61 | T−>C | 1286.37 |
| pSSl688 | −60 | T−>C | 2032.37 |
| pSSl691 | −59 | T−>A | 2005.03 |
| pSSl694 | −58 | C−>A | 1328.70 |
| pSSl697 | −57 | A−>T | 1047.03 |
| pSSl700 | −56 | G−>A | 66.27 |
| pSSl703 | −55 | G−>A | 185.03 |
| pSSl706 | −54 | C−>G | 369.03 |
| pSSl710 | −53 | A−>G | 237.70 |

### III. Preparation of SV40C Promoter

An SV40 derived promoter operably linked to a Site C domain and under the control thereof was prepared. The sequence of the promoter as compared to the TERT minimal promoter is provided below:

```
                        GC-Box GC-Box GC-Box
SV40 ============== ============== ==============
Early GC-Box GC-Box GC-Box
Promoter ============== ============== ==========
AGCAACCATAGTCCCGCCCCTAACTCCGCCCATCCCGCCCCTAACTCCGCCCAGTTCCGCCCATTCTCCGCC
    | || ||||| || | | || || || ||| | | ||| || | |||||
CCCCTCCCGGGTCCCCGGCCCAGCCCCCTCCGGGCCCTCCCAGCCCCTCCCCTTCCTTTCCGCGGCCCCGCC
Telomerase ============== ============== ==========
Minimal GC-Box GC-Box GC-Box
Promoter >
                                                                                                                                    mRNA
                                  Site C mRNA
==== ******************* > > > >
CCATCGCTGAGGCGCGAGTTTCAGGCAGCGCGAGGCCGAGGCCGCCTCGGCCTCTGAGCTATTCCAGAAGTA
| || | ||||||||||||||||||||| | | | | || | || || |
CTCTCCTCGCGGCGCGAGTTTCAGGCAGCGCTGCGTCCTGCTGCGCACGTGGGAAGCCCTGGCCCCGGCCAC
==== *********************
                                  Site C
    > >
mRNA mRNA
```

(SEQ ID NO: 06 & 07)

It has been observed that the Site C sequence causes repression of the SV40 promoter and making a single base change (equivalent to the −65 C-A change) restores full activity of the SV40 promoter.

### IV. Targeting the Therapeutic Effects of the Bax Gene to Cancer Cells Using an Expression Cassette Containing the “SV40C” Promoter

A. Materials and Methods

1. Construction of Recombinant Adenovirus Vectors

Vectors Ad/E1⁻, Ad/CMV-LacZ, Ad/GT-LacZ, Ad/GT-Bax, and Ad/PGK-GV16 are constructed as described previously (Fang B., Ji L., Bouvet M., Roth J. A. Evaluation of GAL4/TATA in vivo. J. Biol. Chem., 273: 4972-4975, 1997; Kagawa S., Pearson S. A., Ji L., Xu K., McDonnell T. J., Swisher S. G., Roth J. A., Fang B. A binary adenoviral vector system for expressing high levels of the proapoptotic gene bax. Gene Ther., 7: 75-79, 2000). Ad/CMV-GFP is provided by Dr. T. J. Liu (M. D. Anderson Cancer Center, Houston, Tex.). Ad/SV40C-LacZ and Ad/hSV40C-GV16 are constructed by replacing the CMV promoter with the Site C containing promoter SV40C as described above. Virus titers are determined by optical absorbance at A₂₆₀ nm (one A₂₆₀ unit=10¹² particles/ml) and by plaque assay. Titers determined by A₂₆₀ (i.e., viral particles) are used in all of the experiments. Particle:plaque ratios normally fall between 30:1 and 100:1. All of the viral preparations are free of contamination by E1⁺ adenovirus and endotoxin.

2. Analysis of in Vitro Gene Expression

Human lung cancer cell lines A549 and H1299 and cervical cancer cell line HeLa are obtained from American Type Culture Collection. Human colon cancer cell lines DLD1 and LoVo are obtained from Dr. T. Fujiwara (Okayama University, Okayama, Japan). NHFB cells and NHBE cells are purchased from Clonetics (San Diego, Calif.) and cultured in media recommended by the manufacturer. Cells are plated 1 day prior to vector infection at densities of 1×10⁵/well in a 24-well plate. Cells are then infected with adenoviral vectors at a MOI of 1000 viral particles/cell. Twenty-four h after infection, cells are either stained with X-Gal to visualize β-galactosidase expression or harvested for biochemical analysis of β-galactosidase activity.

3. Biochemical Analysis

Cultured cells are lysed or tissues from BALB/c mice are homogenized in β-galactosidase assay buffer. Cell or tissue debris is then removed by microcentrifugation. Protein concentrations are determined using a kit from Pierce according to the manufacturer's instructions. β-galactosidase activities are determined using a luminometer and a Galacto-Light Chemiluminescent Assay kit from Tropix, Inc. (Bedford, Mass.).

4. Cell Viability Assay

Cells are plated on 96-well plates at 1×10⁴ per well 1 day prior to virus infection. Cells are then infected with adenoviral vectors at a total MOI of 1500 viral particles/cell. Cells are divided into four groups according to the viral vector system given: Ad/CMV-GFP+Ad/PGK-GV16, Ad/GT-Bax+Ad/CMV-GFP, Ad/GT-Bax+Ad/SV40C-GV16, or Ad/GT-Bax+Ad/PGK-GV1 6. In each group, the ratio of the two viral vectors is 2:1. PBS is used for mock infection. The cell viability is determined by XTT assay using a Cell Proliferation Kit II (Roche Molecular Biochemicals) according to the manufacturer's protocol. In each treatment group, quadruplicate wells are measured for cell viability.

5. Apoptosis Analysis by Flow Cytometry

Cells are plated at densities of 1×10^{6/100}-mm plate 1 day prior to infection. The cells are then infected with recombinant adenoviral vectors at a MOI of 1500 viral particles/cell. Forty-eight h later, both adherent and floating cells are harvested by trypsinization, washed with PBS, and fixed in 70% ethanol overnight. Cells are then stained with propidium iodide for analysis of DNA content. Apoptotic cells are quantified by flow cytometric analysis performed in the Flow Cytometry Core Laboratory at our institution (M. D. Anderson Cancer Center).

6. Animal Experiments

All of the mice are cared for according to the Guide for the Care and Use of Laboratory Animals (NIH publication number 85-23). In vivo infusion of adenoviral vectors into and subsequent tissue removal from BALB/c mice are done as described previously (Fang et al., supra). In the s.c. tumor model, 5×10⁶ H1299 cells are inoculated s.c. into the dorsal flank of 6- to 8-week-old nude mice (Harlan Sprague Dawley, Indianapolis) to establish tumors. After tumors reach 5 mm in diameter, mice are given three sequential intratumoral injections of 9×10¹⁰ viral particles in a volume of 100 μl per dose. Tumor sizes are measured three times a week. Tumor volumes were calculated using the formula a×b²×0.5, where a and b represent the larger and smaller diameters, respectively.

7. Histochemistry Study

For H&E staining, sectioned tissues or tumors are processed as follows. For X-Gal staining, 8-μm-thick frozen sections are fixed with 50% ethanol and 50% methanol for 20 min at −20° C. The fixed sections are then stained with a solution, containing 5 mM K₄Fe(CN)₆, 5 mM K₃Fe(CN)₆, 2 mM MgCl₂, and 1 mg/ml X-Gal, at 37° C. overnight and are finally counterstained with Nuclear Fast Red (Sigma).

8. Analysis of Serum AST and ALT

Blood is drawn from the tail vein of mice 48 h after adenovirus infusion. The levels of serum AST and ALT are measured as described in Kagawa et al., Antitumor effect of adenovirus-mediated Bax gene transfer on p53-sensitive and p53-resistant cancer lines. Cancer Res., 60:1157-1161, 2000.

9. Statistical Analysis

Differences among the treatment groups are assessed by ANOVA using statistical software (StatSoft, Tulsa, Okla.). P≦0.05 is considered significant.

### B. Results

1. Tumor-Specific Transgene Expression Driven by the SV40C Promoter in Vitro

To assess transgene expression from the SV40C promoter in various cells, an adenoviral vector expressing the LacZ gene driven by a SV40C promoter is employed. The SV40C promoter activity is assessed in cultured human lung cancer lines cells (H1299 and A549), colon cancer cells (DLD1 and LoVo), cervical cancer cells (HeLa), NHFB cells, and NHBE cells by infecting the cells at a MOI of 1000 viral particles. Expression of bacterial β-galactosidase is then analyzed 24 h after infection by either X-Gal staining or enzyme assay as described in “Materials and Methods.” In all of the cancer cell lines tested, both the CMV and SV40C promoters drive strong β-galactosidase expression as evidenced by X-Gal staining, whereas in the two normal cell lines, only infection with Ad/CMV-LacZ produces high levels of transgene expression (nearly 100%). Infection of the normal cells at the same MOI with Ad/SV40C-LacZ results in very few LacZ-positive cells. In all of the cells tested, Site promoter activity is significantly higher in cancer cells than in normal cells (P≦0.05). These results together demonstrate that the SV40C promoter is highly active in a variety of cancer cell lines but not in normal cells, indicating that the SV40C promoter is both strong enough and specific enough to be used in targeting transgene expression to tumors.

2. Transcriptional Activity of the SV40C Promoter in Vivo

To investigate the levels of transgene expression induced by the SV40C promoter in vivo, 6×10¹⁰ particles of Ad/SV40C-LacZ, Ad/CMV-LacZ, or Ad/CMV-GFP are infused into BALB/c mice via the tail vein. All of the mice are killed 2 days after vector or PBS infusion; and the liver, spleen, heart, lung, kidney, intestine, ovary, and brain are removed from each for histochemical staining and biochemical analyses of bacterial β-galactosidase expression. High levels of β-galactosidase activity are detected in the livers and spleens of mice treated with Ad/CMV-LacZ. The enzyme activities in other organs of mice treated with Ad/CMV-LacZ are the same as in the background controls. In contrast, the enzyme activities in the livers, spleens, and other organs of mice treated with Ad/SV40C-LacZ are all within the ranges seen in background controls, i.e., PBS- and Ad/CMV-GFP-treated mice. The failure of the SV40C promoter to drive detectable LacZ expression in adult mouse tissues is not attributable to the inability of the SV40C promoter to use the mouse transcriptional machinery, inasmuch as a high level of transgene expression is detected in a mouse lung carcinoma cell line (M109) after infection with Ad/SV40C-LacZ. These findings indicate that the SV40C promoter can be used to prevent transgene expression in normal liver and spleen cells and to minimize the liver and spleen toxicity of a therapeutic gene after its systemic delivery.

3. SV40C Promoter-driven Bax Gene Expression Specifically Suppresses Tumor Cells in Vitro

To test whether the SV40C promoter can be used to negate the toxic effects of the Bax gene on normal cells while preserving its antitumor activity, the recombinant adenoviral vector (Ad/SV40C-GV16) is constructed is described above. The effects of the Bax gene on normal and tumor cells when induced by the SV40C promoter compared with the effects when induced by the PGK promoter are then tested using the binary adenoviral vector system as described in Gu et al., Cancer Res. (2000) 60:5359-64. Human lung cancer lines H1299 and A549, NHBE cells, and NHFB cells are treated with PBS, Ad/CMV-GFP+Ad/PGK-GV16, Ad/GT-Bax+Ad/CMV-GFP, Ad/GT-Bax+Ad/SV40C-GV16, or Ad/GT-Bax+Ad/PGK-GV16. The cells are harvested 48 h after the treatment and subjected to fluorescence-activated cell sorter analysis to determine the fraction of apoptotic cells by quantifying the sub-G, population. Induction of apoptosis in H1299 and A549 cells is comparable after infection with either Ad/GT-Bax+Ad/SV40C-GV16 or Ad/GT-Bax+Ad/PGK-GV16, which suggests that the SV40C promoter is as strong as the PGK promoter in inducing Bax gene expression and apoptosis in tumor cells. In the two normal cell lines (NHBE and NHFB), however, treatment with Ad/GT-Bax+Ad/PGK-GV16 elicits substantial apoptosis as well, whereas treatment with Ad/GT-Bax+Ad/SV40C-GV16 elicits no obvious apoptosis. These results demonstrate that the SV40C promoter can be used to drive tumor-specific proapoptotic gene expression and apoptosis induction while negating the toxicity of a proapoptotic gene to normal cells.

4. Bax Gene Expression Driven by the SV40C Promoter Suppresses Tumor Growth in Vivo

To evaluate the possibility of using the SV40C promoter for in vivo Bax gene therapy, H1299 tumors are established s.c. in nude mice and treated with the Bax gene the expression of which is driven by the SV40C or PGK promoter. After three sequential intratumoral injections of adenoviral vectors, tumor size changes are monitored for 3 weeks. Treatment with Ad/GT-Bax+Ad/SV40C-GV16 or Ad/GT-Bax+Ad/PGK-GV16 results in the same levels of tumor-growth suppression that are significantly different from treatments with PBS, Ad/E1⁻or Ad/GT-LacZ+Ad/SV40C-GV16 groups. These results demonstrate that the hTERT promoter can effectively drive transgene expression in tumors in vivo.

### V. Treatment of Malignant Glioma Cells with the Transfer of Constitutively Active Caspase-6 Using the SV40C Promoter

A. Materials and Methods

1. Cells

Human malignant glioma U87-MG, A172, T98G, and U373-MG cells and human normal fibroblasts MRC5 are purchased from American Tissue Culture Collection (Rockville, Md.). Human malignant glioma GB-1 and U251-MG cells are provided by Dr. Tatsuo Morimura (National Utano Hospital, Kyoto, Japan) and Dr. Akiko Nishiyama (University of Connecticut, Storrs, Conn.), respectively. ALT cell lines (VA13 and SUSM-1) are used as telomerase-independent cell lines. Cells are cultured in DMEM (Life Technologies, Inc.) supplemented with 10% fetal bovine serum (Life Technologies, Inc.), 4 mM glutamine, 100 units/ml penicillin, and 100 μg/ml streptomycin. Human astrocytes TEN are maintained in RPMI 1640 medium (Life Technologies, Inc.) supplemented with 10% fetal bovine serum (Life Technologies, Inc.), 4 mM glutamine, 100 units/ml penicillin, and 100 μg/ml streptomycin. TEN astrocytes are characterized by the presence of the astrocytic marker glial fibrillary acidic protein in nearly 100% of cells when evaluated under immunofluorescent microscope. All of the malignant glioma cells (U87-MG, U251-MG, 25 U373-MG, A172, GB-1, and T98G) are telomerase-positive, whereas TEN, MRC5, VA13, and SUSM-1 cells are telomerase-negative.

2. Construction of the SV40C Promoter Plasmids Carrying Rev-Caspase-6

To construct the rev-caspase-6 expression vector under the SV40C promoter, the SV40C promoter-described above is employed. The CMV promoter-expression vector containing the full-length rev-caspase-6 (pRSC-Rev-caspase-6 or CMV/rev-caspase-6) reported previously (Srinivasula et al. J. Biol. Chem., 273:10107-10111,1998) is used as a template. The 960-bp fragment of rev-caspase-6 is generated by PCR amplification. The sequence of the PCR product is confirmed using ABI PRISM 377 DNA Sequencer system (Applied Biosystems, Foster City, Calif.). The PCR-amplified product is then ligated into the Kasl-Xbal site of pGL3-378 instead of luciferase and designated as the SV40C/rev-caspase-6 expression vector.

3. Transient Transfection Assay

To determine whether the SV40C/rev-caspase-6 construct induces apoptosis only in hTERT-positive cells, transient transfection assays using LipofectAMINE-mediated gene transfer (Life Technologies, Inc.) are performed. The plasmid-expressing GFP, PEGFP-C1 (Clontech, Palo Alto, Calif.), is used as a reporter gene-plasmid. The day before transfection, cells are seeded at 5×10⁴ cells/ml in Lab-Tek chamber slides. The rev-caspase-6 expression vector under the SV40C promoter (SV40C/rev-caspase-6;1 μg) or the CMV promoter (CMV/rev-caspase-6;1 μg) together with pEGFP-C1 (0.3 μg) are transfected into cells and incubated for 48 h. The SV40C/luciferase construct is used as a negative control. To detect the induction of apoptosis, cells are fixed with 1% formaldehyde and 0.2% glutaraldehyde for 5 min, rinsed three times with PBS, and stained with the TUNEL technique (ApopTag Peroxidase In Situ Apoptosis Detection Kit; Intergen, Purchase, N.Y.). Cells are visualized by either bright-field or fluorescence microscopy to detect apoptotic cells or GFP-transfected cells, respectively. An apoptotic index is determined as a percentage of apoptotic cells among 100 GFP-positive cells. For detection of exogenous caspase-6, immunohistochemical staining using antihuman-caspase-6 mouse monoclonal antibody (PharMingen, San Diego, Calif.) is performed instead of TUNEL staining.

4. In Vivo Effect of Rev-Caspase-6 Expression under the SV40C Promoter

Human malignant glioma U87-MG or U373-MG cells (1.0×10⁶ cells in 0.05 ml of serum-free DMEM and 0.05 ml of Matrigel) are inoculated s.c. into the right flank of 8-12-week-old male BALB/c nude mice (six mice for each treatment group), and the tumor growth is monitored using calipers every other day as described previously. When the tumors reach a mean tumor volume of 50-70 mm³, the treatment is initiated to simulate the clinical situation. The SV40C/rev-caspase-6 (10 μg) and cationic lipid (DMRIE; 2 pg; Life Technologies, Inc.) dissolved in 20 μl of sterile PBS are directly injected into the tumor every 24 h for 7 days. The CMV/rev-caspase-6 or SV40C/luciferase construct mixed with DMRIE is used as a positive and negative control, respectively. Mice are sacrificed by cervical dislocation the day after the final treatment. The tumors are removed and frozen rapidly, and 8.0-μm cryosections are made for histological studies. The consecutive sections from treated tumors are used for the TUNEL technique using the ApopTag Peroxidase In Situ Apoptosis Detection Kit and caspase-6 immunohistological staining using anti-caspase-6 antibody as described previously (Kondo et al., Cancer Res., 58: 962-967, 1998.)

B. Results

1. In Vitro Effect of the SV40C/Rev-Caspase-6 on hTERT-Positive or -Negative Cells

To determine whether the SV40C/rev-caspase-6 construct induces apoptosis only in hTERT-positive malignant glioma cells, cells with or without hTERT mRNA are transfected with SV40C/luciferase, SV40C/rev-caspase-6, or CMV/rev-caspase-6 together with the GFP gene (pEGFP-C1). Two days after the transfection, the incidence of apoptosis is determined. U87-MG glioma cells and MRC5 fibroblasts transfected with the SV40C/luciferase construct and pEGFP-C1 retain normal morphology of adherent cells and are TUNEL-negative. Next, U87-MG glioma cells that have the SV40C/rev-caspase-6 vector and pEGFP-C1 display apoptotic morphology and positive staining for TUNEL. In contrast, MRC5 fibroblast cells transfected with the SV40C/rev-caspase-6 and pEGFP-C1 do not undergo apoptosis. Both U87-MG and MRC5 cells undergo apoptosis after transfection with the CMV/rev-caspase-6 construct and pEGFP-C1, respectively. Two days after transfection with the SV40C/rev-caspase-6 vector, apoptosis is induced in 21-54% of malignant glioma cells. The incidence of apoptosis by the SV40C promoter system is similar to that by the CMV-promoter. This finding indicates that apoptosis may be induced in tumor cells once the signals for apoptosis reach a certain critical level. It is found that the apoptosis-induction effect of the SV40C/rev-caspase-6 was specific for hTERT-positive cells. It is also found that induction of apoptosis in hTERT-positive tumor cells is correlated with the activated caspase-6 expression.

2. In Vivo Effect of the SV40C/rev-caspase-6 on Malignant Glioma Cells

To determine the in vivo antitumor effect of the SV40C/rev-caspase-6 construct, hTERT-positive malignant glioma cells are inoculated s.c. in nude mice. After the establishment of s.c. tumors, the SV40C/luciferase (negative control), the SV40C/rev-caspase-6, or the CMV/rev-caspase-6 (positive control; 10 μg each) in the presence of DMRIE (2 μg) is injected directly into tumors every 24 h for 7 days (days 1 to 7). In this experiment, U373-MG cells with high hTERT mRNA expression and U87-MG cells with moderate hTERT mRNA expression are employed. Treatment with the SV40C/rev-caspase-6 construct is found to significantly inhibit the growth of U373-MG s.c. tumors when compared with the SV40C promoter with the luciferase gene (P<0.0005). As predicted from the in vitro experiments, the antitumor effect of SV40C/rev-caspase-6 against U373-MG tumors is not significantly different from that of CMV/rev-caspase-6 (P=0.8561). In the animals treated with the SV40C/rev-caspase-6 construct or CMV/rev-caspase-6, the mean tumor volume on day 8 is reduced by 51% or 52% from the initial tumor size, respectively. In contrast, the mean tumor volume is increased by 39% in control mice treated with the SV40C/luciferase construct. As predicted from the in vitro results, the antitumor effect of CMV/rev-caspase-6 on U87-MG tumors is greater than that of SV40C/rev-caspase-6 (P<0.005). However, the treatment with SV40C/rev-caspase-6 also significantly suppresses the tumor growth compared with the SV40C/luciferase treatment (P<0.005). Significant numbers of apoptotic cells are observed in tumors treated with the SV40C/rev-caspase-6 construct, although tumors treated with control vector (SV40C/luciferase) showed almost no apoptotic cells. The percentage of TUNEL-positive cells is 0.6% or 16.5% in SV40C/luciferase- or SV40C/rev-caspase-6-treated tumors (P=0.0059). The expression of caspase-6 protein is detected throughout the entire tumors treated with the SV40C/rev-caspase-6 construct, whereas few numbers of caspase-6-expressing cells are observed in controls. The percentage of caspase-6-positive cells is 1.4% or 20.6% in SV40C/luciferase- or SV40C/rev-caspase-6-treated tumors (P=0.0482). These results indicate that the cytotoxic effect is mainly attributable to apoptosis induced by expression of caspase-6 protein. It is also found that the effect of SV40C/rev-caspase-6 is more likely to be a robust and durable response rather than a transient response followed by rapid regrowth after the end of treatment.

### VI. SV40C Promoter Induces Tumor-Specific Bax Gene Expression and Cell Killing in Syngenic Mouse Tumor Model and Prevents Systemic Toxicity

SV40C promoter-driven, adenovirus-mediated Bax transgene expression is tested in an established syngenic mouse tumor model and its effects on tumor and normal murine tissues are evaluated. The SV40C promoter is highly active in several murine tumor cell lines and a transformed cell line, but not in non-transformed and normal murine cell lines. The SV40C promoter induces tumor-specific Bax gene expression in mouse UV-2237m fibrosarcoma cells both in vitro and in vivo and suppresses syngenic tumor growth in immune-competent mice with no obvious acute or long-term toxic effects. Moreover, SV40C promoter-driven transgene expression in human CD34(+) bone marrow progenitor cells has effects similar to those observed in other normal human cells, suggesting that the SV40C promoter is much less active in CD34(+) cells than in tumor cells. Together, the findings indicate that the SV40C promoter enables the use of proapoptotic genes for cancer treatment without noticeable effects on progenitor cells.

### VII. FADD Gene Therapy Using the SV40C Promoter to Restrict Induction of Apoptosis to Tumors in Vitro and in Vivo

In this study, the expression vector of FADD gene with death domain operably linked to the SV40C promoter employed in the experiments above (SV40C/FADD) is constructed and investigated for its effect on tumors in vitro and in vivo. Transient transfection with the SV40C/FADD construct induces apoptosis in telomerase-positive tumor cells of wide range. In contrast, normal fibroblast cells without telomerase do not undergo apoptosis following the SV40C/FADD transfer. Furthermore, the growth of subcutaneous tumors in nude mice is significantly suppressed by the intratumoral injection of the SV40C/FADD construct (every day for one week) compared to the control (P<0.0005). The findings described here indicate the high potentiality of a novel telomerase-specific gene therapy of tumors with telomerase.

### VIII. The Telomerase Reverse Transcriptase Promoter Drives Efficacious Tumor Suicide Gene Therapy While Preventing Hepatotoxicity Encountered with Constitutive Promoters

The herpes simplex virus thymidine kinase gene is placed under the control of the SV40C promoter employed above, with the aim of restricting its expression to tumor cells. In transfection experiments, the SV40C promoter driven thymidine kinase gene (SV40Cp/TK) confers ganciclovir sensitivity to all tumor and immortal cell lines tested, whereas normal somatic cells remain largely unaffected. Human SV40Cp/TK-positive cancer cells implanted in nude mice develop into tumors that can be eradicated by ganciclovir treatment. The SV40Cp/TK cassette is inserted into an adenovirus vector and its efficacy in reducing tumor growth is compared with that of an adenovirus carrying the thymidine kinase gene under the control of the cytomegalovirus immediate-early promoter (CMVp/TK). In a xenograft model using the human 143B osteosarcoma cell line, a single injection of either virus results in equivalent tumor regression and survival upon ganciclovir treatment. In animals injected intratumorally with the CMVp/TK adenovirus, expression of the thymidine kinase gene is detected in tumors, as well as in liver samples. Expression of the suicide gene in combination with ganciclovir results in severe liver histopathology and in an elevation of hepatic enzymes. In sharp contrast, when the SV40C promoter controlls the thymidine kinase gene, transgene expression is observed in tumors, but not in liver samples. Normal liver function in these animals is confirmed by serum levels of hepatic enzymes that are indistinguishable from those of control healthy mice. These results indicate that by restricting thymidine kinase expression to tumor cells, the SV40C promoter allows the tumoricidal effect of the suicidal gene to be exerted without detrimental consequences on healthy tissues and vital organs.

### IX. Gene Transfer of Caspase-8 Utilizing the SV40C Promoter

Using the SV40C promoter-driven caspase-8 expression vector (SV40C/caspase-8), apoptosis is restricted to telomerase-positive tumor cells of wide range, and is not seen in normal fibroblast cells without telomerase activity. Furthermore, treatment of subcutaneous tumors in nude mice with the SV40C/caspase-8 construct inhibits tumor growth significantly because of induction of apoptosis (p<0.01).

The above discussion demonstrates that the subject invention provides a safe and effective way to selectively express a protein of interest in telomerase producing/expressing cells, even when such cells are present in a mixed population of cells that do and do not express/produce telomerase. The above discussion also demonstrates that the subject methods have wide application in both therapeutic and diagnostic protocols. With respect to therapeutic protocols, advantages of the subject invention include the ability to limit any treatment agents to contact with disease cells, thereby increasing effectiveness and decreasing toxicity. With respect to diagnostics, one advantage is the ability to perform in vivo testing without removing a sample from the host. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of expressing a protein in a telomerase expressing cell, said method comprising;
introducing into said cell an effective amount of an expression cassette comprising a coding sequence for said protein operably linked to a promoter and a Site C repressor site to produce said protein in said cell, wherein said expression cassette does not include the TERT minimal promoter.

2. The method according to claim 1, wherein said Site C repressor site comprises a sequence that is substantially the same as or identical to the sequence of SEQ ID NO: 01.

3. The method according to claim 1, wherein said coding sequence for said protein encodes a therapeutic protein.

4. The method according to claim 3, wherein said therapeutic protein is a toxin.

5. The method according to claim 3, wherein said therapeutic protein is an enzyme.

6. The method according to claim 1, wherein said coding sequence for said protein encodes a marker protein.

7. The method according to claim 6, wherein said marker protein is an enzyme.

8. The method according to claim 6, wherein said marker protein is a fluorescent protein.

9. The method of claim 1, wherein said cell is an abnormally proliferating cell.

10. The method according to claim 1, wherein said promoter is not a Tert promoter.

11. A method of selectively expressing a protein in telomerase expressing cells present in a population of cells that includes cells that do not express telomerase, said method comprising:
introducing into said cells of said population an effective amount of an expression cassette comprising a coding sequence for said protein operably linked to a Site C repressor site and a promoter to selectively produce said protein in said telomerase expressing cells of said population of cells, wherein said expression cassette does not include the TERT minimal promoter.

12. The method according to claim 11, wherein said Site C repressor site comprises a sequence that is substantially the same as or identical to the sequence of SEQ ID NO: 01.

13. The method according to claim 11, wherein said coding sequence for said protein encodes a therapeutic protein.

14. The method according to claim 13, wherein said therapeutic protein is a toxin.

15. The method according to claim 14, wherein said therapeutic protein is an enzyme.

16. The method according to claim 11, wherein said coding sequence for said protein encodes a marker protein.

17. The method according to claim 16, wherein said marker protein is an enzyme.

18. The method according to claim 16, wherein said marker protein is a fluorescent protein.

19. The method according to claim 11, wherein said promoter is not a Tert promoter.

20. A method of selectively expressing a protein in telomerase expressing cells present in a multicellular organism which includes cells that do not express telomerase, said method comprising:
administering to said organism an effective amount of an expression cassette comprising a coding sequence for said protein operably linked to a Site C repressor site and a promoter to selectively produce said protein in said telomerase expressing cells of said multicellular organism, wherein said expression cassette does not include the TERT minimal promoter.

21. The method according to claim 20, wherein said Site C repressor site comprises a sequence that is substantially the same as or identical to the sequence of SEQ. ID NO: 1.

22. The method according to claim 20, wherein said coding sequence for said protein encodes a therapeutic protein.

23. The method according to claim 22, wherein said therapeutic protein is a toxin.

24. The method according to claim 23, wherein said therapeutic protein is an enzyme.

25. The method according to claim 20, wherein said coding sequence for said protein encodes a marker protein.

26. The method according to claim 25, wherein said marker protein is an enzyme.

27. The method according to claim 26, wherein said marker protein is a fluorescent protein.

28. The method according to claim 20, wherein said multicellular organism is a mammal.

29. The method according to claim 28, wherein said mammal is a human.

30. The method according to claim 20, wherein said promoter is not a Tert promoter.

31. A method of diagnosing the presence of telomerase expressing cells in a host, said method comprising:
administering to said host an effective amount of an expression cassette comprising a coding sequence for a marker protein operably linked to a Site C repressor site and a promoter to selectively produce said marker protein in said telomerase expressing cells of said host, wherein said expression cassette does not include the TERT minimal promoter; and
detecting the presence of said marker protein in said host to diagnose the presence of telomerase expressing cells in said host.

32. The method according to claim 31, wherein said marker protein is an enzyme.

33. The method according to claim 31, wherein said marker protein is a fluorescent protein.

34. The method according to claim 31, wherein said host is a mammal.

35. The method according to claim 34, wherein said mammal is a human.

36. The method according to claim 31, wherein said telomerase expressing cells are abnormally proliferative cells.

37. The method according to claim 31, wherein said promoter is not a Tert promoter.

38. A method of treating a host for a cellular proliferative disease, said method comprising:
administering to said host an effective amount of an expression cassette comprising a coding sequence for a therapeutic protein operatively linked to a Site C repressor site and a promoter to selectively produce said therapeutic protein in said telomerase expressing cells of said host, wherein said expression cassette does not include the TERT minimal promoter.

39. The method according to claim 38, wherein said therapeutic protein is a toxin.

40. The method according to claim 38, wherein said therapeutic protein is an enzyme.

41. The method according to claim 38, wherein said host is a mammal.

42. The method according to claim 38, wherein said mammal is a human.

43. The method according to claim 38, wherein said promoter is not a Tert promoter.

44. An expression cassette comprising:
(a) a Site C repressor site;
(b) a promoter; and
(c) a DNA nucleotide coding sequence encoding a protein of interest;
wherein said expression cassette does not include the TERT minimal promoter.

45. The expression cassette of claim 44, wherein said Site C repressor site has a sequence that is substantially the same as or identical to a sequence selected from the group consisting of SEQ ID NOs: 01 to 04.

46. The expression cassette according to claim 44, wherein said protein is a therapeutic protein.

47. The expression cassette according to claim 46, wherein said therapeutic protein is an enzyme.

48. The expression cassette according to claim 46, wherein said therapeutic protein is a toxin.

49. The expression cassette according to claim 44, wherein said protein is a marker protein.

50. The expression cassette according to claim 49, wherein said marker protein is an enzyme.

51. The expression cassette according to claim 49, wherein said marker protein is a fluorescent protein.

52. The expression cassette according to claim 44, wherein said promoter is not a Tert promoter.

53. A vector comprising the expression cassette of claim 44.

54. A vector according to claim 53, wherein said vector is a viral vector.

55. A vector according to claim 54, wherein the viral vector is a retrovirus vector, an adenovirus vector, an adeno-associated virus vector, a vaccinia virus vector, a herpes virus vector or a rabies virus vector.

56. A vector according to claim 53, wherein the vector is a non-viral vector.

57. A vector according to claim 56, wherein the vector is a plasmid.

58. A cell comprising an expression cassette according to claim 44.

59. An expression cassette comprising:
(a) a Site C repressor site;
(b) a promoter; and
(c) a nucleic acid insertion site comprising at least one restriction endonuclease recognized sequence;
wherein said expression cassette does not include a TERT minimal promoter.

60. The expression cassette according to claim 59, wherein said nucleic acid insertion site is a multiple cloning site.

61. The expression cassette according to claim 59, wherein said promoter is not a Tert promoter.

62. A kit for use in expressing a protein in a telomerase expressing cell, said kit comprising:
(a) an expression cassette according to claim 44; and
(b) instructions for using said kit.

63. A kit for use in expressing a protein in a telomerase expressing cell, said kit comprising:
(a) an expression cassette according to claim 59; and
(b) at least one of:
(i) a restriction endonuclease that cuts said restriction endonuclease recognized sequence; and
(ii) a nucleic acid comprising a coding sequence for a protein flanked on either side by said restriction endonuclease recognized sequence.
